(19) United States
(12) Patent Application Publication
(10) Pub. No.: US 2008/0183172 A1
(43) Pub. Date: Jul. 31, 2008

(54) RETENTION FEATURE FOR PLATE GUIDES

(75) Inventor: Daniel D. Fritzinger, Warsaw, IN (US)

Correspondence Address: ZIMMER TECHNOLOGY - BAKER & DANIELS, 111 EAST WAYNE STREET, SUITE 800, FORT WAYNE, IN, 46802, US

(73) Assignee: ZIMMER TECHNOLOGY, INC., Warsaw, IN (US)

(21) Appl. No.: 11/627,531

(22) Filed: Jan. 26, 2007

(51) Int. Cl. A61B 17/58; A61B 17/56

(52) U.S. Cl. 606/71; 606/280; 606/70; 606/301

(57) ABSTRACT

A plate guide including a body having an upper surface, a lower surface, and a projection extending therefrom. The projection may be configured to be received within an aperture in a bone plate. In one exemplary embodiment, the aperture in the bone plate is a bone screw receiving aperture. In another exemplary embodiment, the projection includes a resiliently deformable finger. The resiliently deformable finger may provide a friction fit with the aperture in the bone plate to secure the plate guide in a substantially fixed position relative to the bone plate.

### BACKGROUND

1. Field of the Invention

The present invention relates to plate guides, and, more particularly, to a mechanism for securing plate guides to bone plates.

2. Description of the Related Art

Orthopedic bone plates may be used to maintain different parts of a fractured bone substantially stationary relative to one another. A bone plate may be formed as an elongate body having bone screw receiving apertures extending therethrough and may be positioned to extend across a fracture line in a bone. Once positioned, cerclage wire may be placed over the bone plate to temporarily secure the bone plate to the fragments of bone. Corresponding bone screws may then be inserted through the bone screw receiving apertures in the bone plate to secure the bone plate in position on the bone.

Due to the contour of the bone against which the bone plate is positioned, the bone plate may be configured with bone screw receiving apertures having varying angular alignments. Thus, it may take significant time for a surgeon to colinearly align a drill bit with the bone screw receiving apertures to drill pilot holes in the bone for the receipt of corresponding bone screws. To facilitate the proper alignment of the pilot holes with the apertures in the bone plate, a plate guide may used. The plate guide may be positioned adjacent the bone plate to colinearly align cannulas held by the plate guide with the bone screw receiving apertures of the bone plate. The cannulas may then be used by a surgeon as a guide to form pilot holes in the bone having the same angular alignment as the bone screw receiving apertures.

### SUMMARY

The present invention relates to plate guides, and, more particularly, to a mechanism for securing plate guides to bone plates. In one embodiment, a plate guide includes a body having an upper surface, a lower surface, and a projection extending therefrom. The projection is configured to be received within an aperture in a bone plate. In one exemplary embodiment, the aperture in the bone plate is a bone screw receiving aperture. In another exemplary embodiment, the projection includes a resiliently deformable finger. The resiliently deformable finger may provide a friction fit with the aperture in the bone plate to secure the plate guide in a substantially fixed position relative to the bone plate.

Advantageously, the use of a projection configured to be received in a bone screw receiving aperture allows for the plate guide of the present invention to be utilized with any existing bone plate having a bone screw receiving aperture. Thus, the need to machine a custom aperture in the bone plate to receive the projection is eliminated, reducing manufacturing costs. Additionally, even if a custom aperture is formed in a bone plate, the use of a projection including a resiliently deformable finger for mating with the aperture in the bone plate provides added retention of the plate guide on the bone plate by providing a friction fit.

Moreover, the plate guide of the present invention may be attached to any size of bone plate. In contrast, a thumbscrew, for example, configured for receipt in a custom aperture of a bone plate to retain a plate guide thereon must be large enough to facilitate grasping and manipulation by a surgeon. Additionally, the thumbscrew must also be small enough that the custom aperture formed in the bone plate does not significantly lessen the integrity of the bone plate. The use of a projection configured for receipt in an existing bone screw receiving aperture of a bone plate eliminates these concerns. Specifically, the integrity of the bone plate is not compromised, as the bone plate was previously engineered to include the bone screw receiving aperture. Additionally, in contrast to a thumbscrew design, a surgeon does not have to manipulate the projection, but can insert the projection by grasping and manipulating the body of the plate guide.

In one form thereof, the present invention provides an orthopedic system, including a plate guide configured to be connected to an orthopedic bone plate, the plate guide comprising a body having an upper surface, a lower surface, and a channel extending between the upper surface and the lower surface, and a projection extending from the lower surface of the body, the projection adapted to be received within a first bone screw receiving aperture formed in the orthopedic bone plate, whereby receipt of the projection within the first bone screw receiving aperture in the orthopedic bone plate colinearly aligns at least one of the channels of the plate guide with a second bone screw receiving aperture formed in the orthopedic bone plate.

In another form thereof, the present invention provides an method for attaching an orthopedic bone plate to a bone, including the steps of positioning an orthopedic bone plate having a plurality of bone screw receiving apertures adjacent a bone, seating a plate guide to the orthopedic bone plate via one of the plurality of bone screw receiving apertures, removing the plate guide from the orthopedic bone plate, securing the orthopedic bone plate to a bone via one of the plurality of bone screw receiving apertures.

In yet another form thereof, the present invention provides an orthopedic system, including a orthopedic bone plate having an aperture formed therein, a plate guide having a plurality of channels formed therein and a projection extending therefrom, the projection including a pair of resiliently deformable fingers configured for receipt within the aperture of the orthopedic bone plate, whereby the resiliently deformable fingers of the projection form a friction fit between the plate guide and the orthopedic bone plate, and a plurality of cannulas configured for receipt with the plurality of channels formed in the plate guide.

In yet another form thereof, the present invention provides an orthopedic system, including a plate guide configured to be connected to an orthopedic bone plate, the plate guide including a body having an upper surface, a lower surface, and a channel extending between the upper surface and the lower surface, and attachment means for selectively attaching the plate guide to an orthopedic bone plate via a bone screw receiving aperture formed in the orthopedic bone plate, whereby receipt of the attachment means within the first bone screw receiving aperture in the orthopedic bone plate colinearly aligns at least one of the channels of the plate guide with a second bone screw receiving aperture formed in the orthopedic bone plate.

### BRIEF DESCRIPTION OF THE DRAWINGS

The above-mentioned and other features and advantages of this invention, and the manner of attaining them, will become more apparent and the invention itself will be better understood by reference to the following description of an embodiment of the invention taken in conjunction with the accompanying drawings, wherein:

FIG. 1 is a perspective view of a bone plate, a plate guide, and a plurality of cannulas received by the plate guide;

FIG. 2 is a perspective view of a plate guide having an undersized boss extending therefrom;

FIG. 3 is a bone plate having a custom aperture formed therein;

FIG. 4 is a perspective view of a plate guide according to one embodiment of the present invention;

FIG. 5 is a rear view of the plate guide of FIG. 4;

FIG. 6 is a cross sectional view of the plate guide of FIG. 4 taken along line 6-6 of FIG. 4;

FIG. 7 is an enlarged partial cross sectional view taken along dashed line 7-7 of FIG. 6; and

FIG. 8 is a perspective view of the bottom of a bone plate depicting the plate guide of FIG. 4 connected thereto.

Corresponding reference characters indicate corresponding parts throughout the several views. The exemplification set out herein illustrates a preferred embodiment of the invention, in one form, and such exemplification is not to be construed as limiting the scope of the invention in any manner.

### DETAILED DESCRIPTION

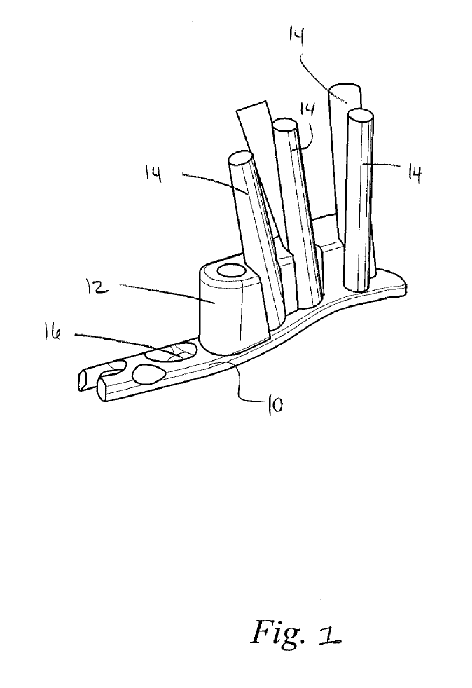

FIG. 1 depicts bone plate 10 having plate guide 12 positioned adjacent thereto. Received within channels formed in plate guide 12 are cannulas 14. Plate guide 12 is positioned on bone plate 10 to align cannulas 14 with bone screw receiving apertures 16 formed in bone plate 10. As used herein a “bone screw receiving aperture” is an aperture formed in a bone plate which is sized to accommodate and cooperate with a corresponding bone screw. For the purposes of this document, a “corresponding bone screw” is a bone screw configured to be received in a bone screw receiving aperture of a bone plate and retain the bone plate in a desired position on a bone when received in a bone screw receiving aperture of the bone plate, i.e., is of sufficient strength to retain the bone plate in the desired position on the bone during physiological loading and, in the case of an articulating bone, during articulation. A “corresponding bone screw” also includes threads having a profile, shape, and/or pitch designed to securely engage cancellous and/or cortical bone.

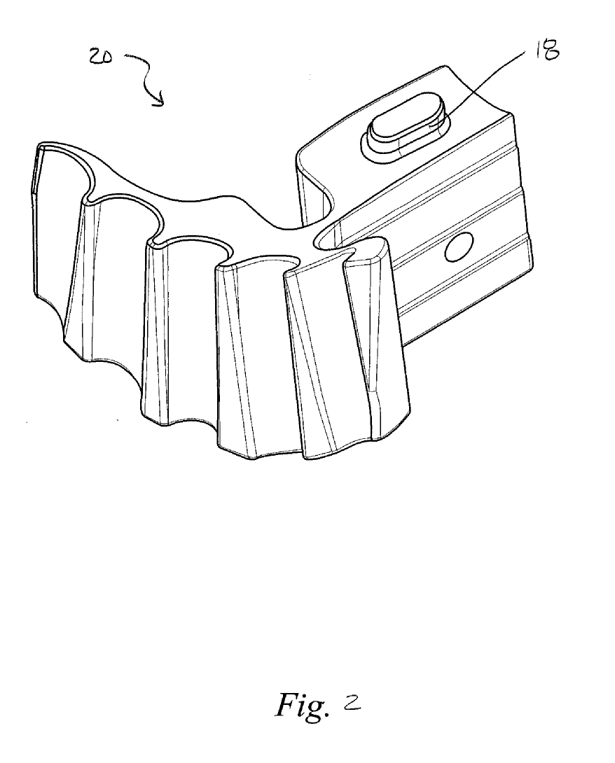

Referring to FIG. 2, to properly position a plate guide on a bone plate, boss 18 may be used. Boss 18 is depicted on a second plate guide 20, which is similar to the plate guide disclosed in U.S. patent application Ser. No. 11/224,686, entitled BONE FRACTURE FIXATION SYSTEM, which was filed on Oct. 6, 2005, the entire disclosure of which is expressly incorporated by reference herein. Boss 18 of plate guide 20 is configured to be receiving in a custom aperture machined in a corresponding bone plate, such as bone plate 10. Thus, the use of boss 18 increases the cost of manufacturing the bone plate due to the additional machining steps required to form the custom aperture. Additionally, to allow for boss 18 to be easily inserted within the custom aperture of a bone plate, boss 18 is undersized with respect to the custom aperture of the corresponding bone plate. This design allows slight movement of boss 18 relative to the corresponding bone plate and prevents boss 18 from securely attaching plate guide 20 thereto. As a result, boss 18 functions solely as a locating device for generally locating the proper position of plate guide 20 on the corresponding bone plate.

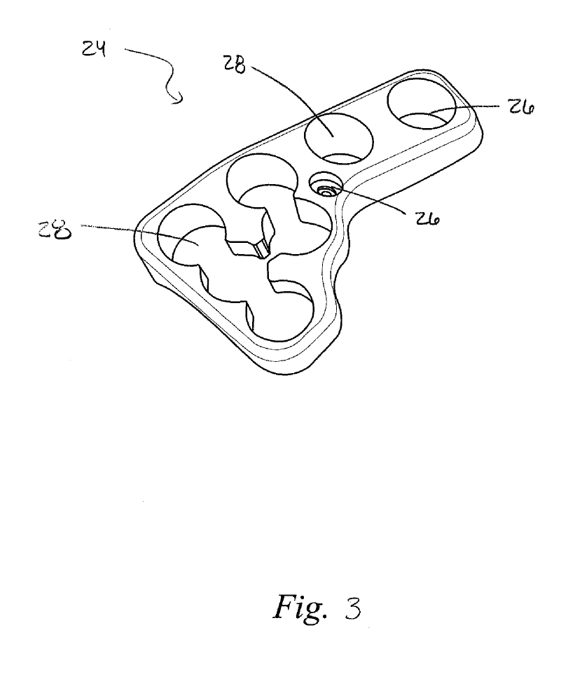

Depicted in FIG. 3 is bone plate 24. Bone plate 24 includes custom aperture 26 and bone screw receiving apertures 28 extending therethrough. Bone plate 24 is configured for attachment to a plate guide (not shown) having an aperture formed therein similar to aperture 26 of bone plate 24. Specifically, when custom aperture 26 and the corresponding aperture of the plate guide are aligned, a thumbscrew may be inserted through the aperture of the plate guide and into custom aperture 26 of bone plate 24 to attach the plate guide to bone plate 24. As discussed above, the use of custom aperture 26 to retain a plate guide on bone plate 24 requires machining custom aperture 26 for the receipt of a thumbscrew. Additionally, as discussed herein above, when a thumbscrew is used to secure a plate guide to a bone plate, the thumbscrew must be large enough to facilitate grasping and manipulation by a surgeon. However, the thumbscrew must also be small enough that the custom aperture formed in the bone plate does not significantly lessen the integrity of the bone plate.

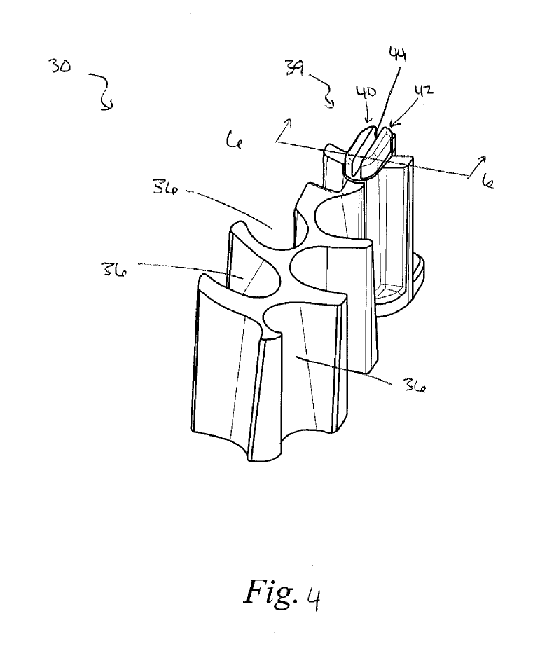

FIG. 4 depicts plate guide 30 according to the present invention. Plate guide 30 includes body 31 having lower surface 32 and upper surface 34. Lower surface 32 may be configured to engage the surface of a bone plate, such as bone plate 58 of FIG. 8. Additionally, extending between lower surface 32 and upper surface 34 are a plurality of channels 36. Channels 36 are configured to receive cannulas 38 (FIG. 8), as described in detail below. While described and depicted herein as partially surrounding cannulas 38, channels 36 may be formed to plate guide 30 to substantially entirely surround cannulas 38. Extending from lower surface 32 of plate guide 30 is projection 39. Projection 39 of plate guide 30 is configured for receipt within of bone screw receiving apertures 60 of bone plate 58, shown in FIG. 8. While depicted herein as a fibular bone plate, bone plate 58 may be any bone plate configured to be positioned on and secured to a corresponding bone. Similarly, while bone plate 30 is depicted herein as a fibular plate guide, plate guide 30 may be configured for use with any type of bone plate.

Referring to FIG. 8, bone plate 58 includes a plurality of bone screw receiving apertures 60 and other, non bone screw receiving apertures 62 extending through bone plate 58. Bone screw receiving apertures 60 may be threaded, non-threaded, or a combination of threaded and non-threaded depending on the corresponding bone screw intended to be received therein. Bone screw receiving apertures 60 and non bone screw receiving apertures 62 extend from upper surface 64 to lower, bone engaging surface 66 of bone plate 58. Separating upper surface 64 from lower surface 66 is perimeter wall 67. Projection 39 is configured for receipt within one of bone screw receiving apertures 60 of bone plate 58. Thus, by retaining plate guide 30 on bone plate 58 through a preexisting bone screw receiving aperture 60, the need to machine a custom aperture in bone plate 58 is eliminated. Additionally, the use of projection 39 to attach plate guide 30 to bone plate 58, allows for plate guide 30 to be modified for use with any size of bone plate 58. Thus, the use of plate guide 30 with smaller sized bone plates 58 is envisioned, as projection 39 eliminates the need for a custom aperture in the smaller sizes of bone plates 58 and helps to maintain the integrity of the smaller sized bone plates 58, as described above.

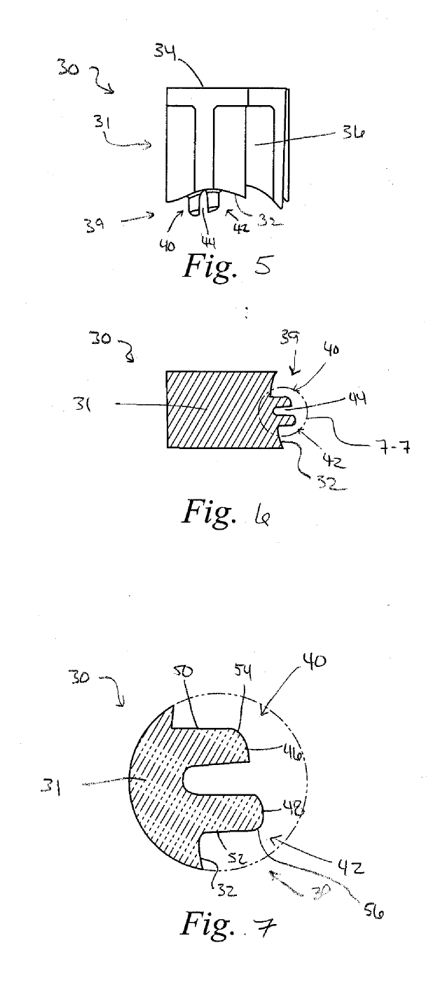

In one exemplary embodiment, projection 39 is defined by resiliently deformable fingers 40, 42, as shown in FIGS. 4-8. Extending between resiliently deformable fingers 40, 42 is slot 44. Slot 44 allows for resiliently deformable fingers 40, 42 to be pressed inward, toward one another. In this embodiment, fingers 40, 42 provide for a friction fit between projection 39 and the walls defining bone screw receiving apertures 60 of bone plate 58, as shown in FIG. 8. Specifically, with reference to FIG. 8, when projection 39 received within one of bone screw receiving apertures 60 of bone plate 58, fingers 40, 42 are pressed inward, causing fingers 40, 42 to exert an outward force resulting in fingers 40, 42 engaging the wall defining the one of bone screw receiving apertures 60. This force is sufficient to retain plate guide 30 to bone plate 58 and substantially prevent movement of plate guide 30 relative to bone plate 58.

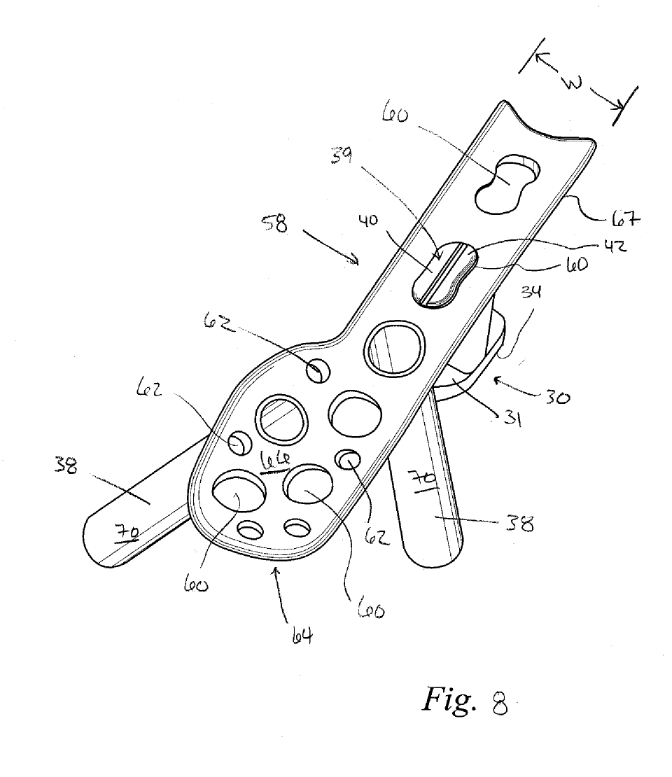

Additionally, as shown in FIG. 7, fingers 40, 42 include ends 46, 48 separated from side walls 50, 52 by tapered edges 54, 56. Tapered edges 54, 56 facilitate insertion of resiliently deformable fingers 40, 42 into one of bone screw receiving apertures 60 of bone plate 58. Specifically, tapered edges 54, 56 allow for ends 46, 48 to be positioned within one of bone screw receiving apertures 60 of bone plate 58 and, as fingers 40, 42 are advanced into one of bone screw receiving apertures 60, tapered edges 54, 56 guide fingers 40, 42 into the same. In another exemplary embodiment, fingers 40, 42 may be configured so that an audible sound is made when projection 39 is properly seated within one of bone screw receiving apertures 60 of bone plate 58. For example, a fingers 40, 42 may include a detent mechanism which interacts with lower surface 66 of bone plate 58. In this embodiment, the audible sound provides feedback to a surgeon indicating that plate guide 30 is properly positioned and retained on bone plate 58.

In another exemplary embodiment, projection 39 is formed by a rigid finger (not shown) and a resiliently deformable finger, such as finger 40. In this embodiment, the resiliently deformable finger provides for a friction fit between projection 39 and the wall defining one of bone screw receiving apertures 60 of bone plate 58. In another exemplary embodiment, projection 39 includes at least one of fingers 40, 42, which is configured for use with a non bone screw receiving hole 62 of bone plate 58. In yet another exemplary embodiment, a custom aperture may be formed in bone plate 58 for the receipt of projection 39, which may including at least one of fingers 40, 42, to position and retain plate guide 30 on bone plate 58. In yet another exemplary embodiment, fingers 40, 42 may be separated by a greater distance than the embodiment of FIGS. 4-8 by a wider slot 44. In this embodiment, the separation between finger 40 and finger 42 may be substantially equal to the width W (FIG. 8) of bone plate 58. Additionally, perimeter wall 67 of bone plate 58 may also include indentations (not shown) for the receipt of fingers 40,42 therein. In this manner, plate guide 38 may be connected to bone plate 58 along perimeter wall 67.

Referring to FIG. 8, to secure plate guide 30 to bone plate 58, projection 39 of plate guide 30 is positioned adjacent one of bone screw receiving apertures 60 formed in bone plate 58. Projection 39 is then seated in the one of bone screw receiving apertures 60, as described in detail above. Projection 39 of plate guide 30 may be seated to bone plate 58 before or after bone plate 58 is positioned adjacent a bone. Once properly seated, projection 39 substantially prevents movement of plate guide 30 with respect to bone plate 58. Cannulas 38 are then positioned within channels 36 (FIG. 4) of plate guide 30 and guided toward respective bone screw receiving apertures 60. Specifically, channels 36 of plate guide 30 are configured to colinearly align cannulas 38 with the angular alignment of respective bone screw receiving apertures 60, which, due to the contour of the bone against which bone plate 58 is positioned, may varying. Stated another way, cannulas 38 are colinear with the longitudinal axis that would be formed by a screw seated in respective bone screw receiving apertures 60. In one exemplary embodiment, bone screw receiving apertures 60 are threaded and cannulas 38 include outer surface 70 which is partially threaded to threadingly engage threaded bone screw receiving apertures 60. The threading engagement between cannulas 38 and bone screw receiving apertures 60 further secure cannulas 38 to bone plate 58.

Apertures 68 extend through cannulas 38 and allow a surgeon to pass a drill through cannulas 38 and bone screw receiving apertures 60 to drill pilot holes for corresponding bone screws, for example. Due to the use of plate guide 30, the angular orientation of the pilot holes drilled by the surgeon through apertures 68 will be substantially substantially colinear with the angular orientation of the bone screw receiving apertures 60, as discussed above. Once the pilot holes have been drilled, cannulas 38 may be removed from plate guide 30 and plate guide 30 may be removed from bone screw receiving aperture 60 of bone plate 58. With plate guide 30 removed, a surgeon may insert corresponding bone screws through the bone screw receiving apertures and into the previously drilled pilot holes to retain bone plate 58 in the desired position on the bone.

While this invention has been described as having a preferred design, the present invention can be further modified within the spirit and scope of this disclosure. This application is therefore intended to cover any variations, uses, or adaptations of the invention using its general principles. Further, this application is intended to cover such departures from the present disclosure as come within known or customary practice in the art to which this invention pertains and which fall within the limits of the appended claims.

What is claimed is:

1. An orthopedic system, comprising:
a plate guide configured to be connected to an orthopedic bone plate, the plate guide comprising:
a body having an upper surface, a lower surface, and a channel extending between said upper surface and said lower surface; and
a projection extending from said lower surface of said body, said projection adapted to be received within a first bone screw receiving aperture formed in the orthopedic bone plate, whereby receipt of said projection within the first bone screw receiving aperture in the orthopedic bone plate colinearly aligns at least one of said channels of said plate guide with a second bone screw receiving aperture formed in the orthopedic bone plate.

2. The orthopedic system of claim 1, wherein said projection further comprises a resiliently deformable finger.

3. The orthopedic system of claim 1, further comprising a cannula sized for receipt within said channel of said plate guide.

4. The orthopedic system of claim 1, wherein the orthopedic bone plate is a fibular bone plate.

5. The orthopedic system of claim 1, wherein said plate guide is a fibular plate guide.

6. An method for attaching an orthopedic bone plate to a bone, comprising the steps of:
positioning an orthopedic bone plate having a plurality of bone screw receiving apertures adjacent a bone;
seating a plate guide to the orthopedic bone plate via one of the plurality of bone screw receiving apertures;
removing the plate guide from the orthopedic bone plate;
securing the orthopedic bone plate to a bone via one of the plurality of bone screw receiving apertures.

7. The method for attaching an orthopedic bone plate to a bone of claim 6, wherein said securing step further comprises inserting a corresponding bone screw through the bone screw receiving aperture.

8. The method for attaching an orthopedic bone plate to a bone of claim 6, wherein the plate guide includes a channel formed therein, the method further comprising the step of positioning a cannula in the channel formed in the plate guide.

9. The method for attaching an orthopedic bone plate to a bone of claim 8, further comprising the step of guiding a drill through said cannula to form a pilot hole in the bone.

10. The method for attaching an orthopedic bone plate to a bone of claim 6, wherein the orthopedic bone plate is a fibular bone plate.

11. An orthopedic system, comprising:
a orthopedic bone plate having an aperture formed therein;
a plate guide having a plurality of channels formed therein and a projection extending therefrom, said projection including a pair of resiliently deformable fingers configured for receipt within said aperture of said orthopedic bone plate, whereby said resiliently deformable fingers of said projection form a friction fit between said plate guide and said orthopedic bone plate; and
a plurality of cannulas configured for receipt with said plurality of channels formed in said plate guide.

12. The orthopedic system of claim 11, wherein said orthopedic bone plate comprises a fibular bone plate.

13. The orthopedic system of claim 11, wherein said aperture in said orthopedic bone plate comprises a bone screw receiving aperture.

14. An orthopedic system, comprising:
a plate guide configured to be connected to an orthopedic bone plate, the plate guide comprising:
a body having an upper surface, a lower surface, and a channel extending between said upper surface and said lower surface; and
attachment means for selectively attaching said plate guide to an orthopedic bone plate via a bone screw receiving aperture formed in the orthopedic bone plate, whereby receipt of said attachment means within the first bone screw receiving aperture in the orthopedic bone plate colinearly aligns at least one of said channels of said plate guide with a second bone screw receiving aperture formed in the orthopedic bone plate.

15. The orthopedic system of claim 14, further comprising a cannula sized for receipt within said channel of said plate guide.

16. The orthopedic system of claim 14, wherein said plate guide is a fibular plate guide.

17. The orthopedic system of claim 14, wherein the orthopedic bone plate is a fibular bone plate.
